SEQUENCE LISTING (1 sequence)

SEQ ID NO: 1 - protein, 10 aa, Homo sapiens
SAVMALQEAC

(19) United States
(12) Patent Application Publication
(10) Pub. No.: US 2010/0240054 A1
(43) Pub. Date: Sep. 23, 2010

(54) IDENTIFICATION AND ISOLATION OF FETAL CELLS AND NUCLEIC ACID

(75) Inventor: Farideh Z. BISCHOFF, Sugar Land, TX (US)

Correspondence Address: COOLEY LLP;ATTN: Patent Group, Suite 1100, 777 - 6th Street, NW, WASHINGTON, DC, 20001, US

(73) Assignee: BIOCEPT, INC., San Diego, CA (US)

(21) Appl. No.: 12/725,331

(22) Filed: Mar. 16, 2010

(30) Foreign Application Priority Data
Sep. 22, 2008 (US) PCT/US08/77251

(51) Int. Cl. C12Q 1/68; C07K 16/18; G01N 33/50; C07H 21/04; C09K 3/00

(52) U.S. Cl. 435/6; 530/387.1; 530/387.3; 436/94; 536/25.4; 252/182.12

(57) ABSTRACT

The present invention provides methods, antibodies and kits useful for detecting the presence of a fetal cell and/or fetal nucleic acids in a biological sample obtained from a maternal host. It also provides methods and kits for isolating fetal nucleic acid from maternal cervical mucus samples, and for testing or screening the isolated fetal nucleic acid for genetic abnormalities in fetuses.

The present application claims priority to U.S. Provisional Application No. 60/974,392, filed Sep. 21, 2007, which is incorporated by reference herein in its entirety.

### BACKGROUND OF THE INVENTION

Prenatal testing or screening is usually performed to determine the gender of the fetus or to detect genetic disorders and/or chromosomal abnormalities in the fetus during pregnancy. As of today, over 4000 genetic disorders, caused by one or more faulty genes, have been recognized. Some examples include Cystic Fibrosis, Huntington's Disease, Beta Thalassaemia, Myotonic Dystrophy, Sickle Cell Anemia, Porphyria, and Fragile-X-Syndrome. Chromosomal abnormality is caused by aberrations in chromosome numbers, duplication or absence of chromosomal material, and by defects in chromosome structure. Examples of chromosomal abnormalities are trisomies, e.g., trisomy 16, a major cause of miscarriage in the first trimester, trisomy 21 (Down syndrome), trisomy 13 (Patau syndrome), trisomy 18 (Edwards syndrome), Klinefelter's syndrome (47, XXY), (47, XYY), and (47, XXX); the absence of chromosomes (monosomy), e.g., Turner syndrome (45, X0); chromosomal translocations, deletions and/or microdeletions, e.g., Robertsonian translocation, Angelman syndrome, DiGeorge syndrome and Wolf-Hirschhorn Syndrome.

Currently available prenatal genetic tests usually involve invasive procedures. For example, chorionic villus sampling (CVS) performed on a pregnant woman around 10-12 weeks into the pregnancy and amniocentesis performed at around 14-16 weeks all contain invasive procedures to obtain the sample for testing chromosomal abnormalities in a fetus. Fetal cells obtained via these sampling procedures are usually tested for chromosomal abnormalities using cytogenetic or fluorescent in situ hybridization (FISH) analyses.

While these procedures can be useful for detecting chromosomal aberrations, they have been shown to be associated with the risk of miscarriage. Therefore amniocentesis or CVS is only offered to women perceived to be at increased risk, including those of advanced maternal age (>35 years), those with abnormal maternal serum screening or those who have had a previous fetal chromosomal abnormality. As a result of these tests the percentage of women over the age of 35 who give birth to babies with chromosomal aberrations such as Down syndrome has drastically reduced. However, lack of appropriate or relatively safe prenatal testing or screening for the majority of pregnant women has resulted in about 80% of Down syndrome babies born to women under 35 years of age.

Thus there is a need for non-invasive screening tests for the general population of pregnant women, especially tests directed to identifying fetal chromosomal aberrations as well as other genetic variations or defects. This requires non-invasive techniques of detecting, as well as isolating fetal cells and fetal nucleic acid that can be used for prenatal genetic screening.

### SUMMARY OF THE INVENTION

The present invention is based, in part, on the discovery that nucleosome epitopes can be used as markers for fetal cells or fetal nucleic acids. Accordingly, the present invention provides antibodies to these nucleosome epitopes, kits containing such antibodies, and methods for using such antibodies to detect or isolate fetal cells and/or fetal nucleic acids.

In one embodiment of the invention, it provides an antibody that specifically binds to a fetal nucleosome epitope. In another embodiment of the invention, it provides a kit comprising an antibody of the invention.

In yet another embodiment of the invention, it provides a kit suitable for testing the genetic composition of a fetus. The kit comprises an isolated nucleic acid sample of the fetus that is isolated by using an antibody of the invention.

In still another embodiment of the invention, it provides a method for detecting the presence of fetal nucleic acids and/or fetal cells in a biological sample obtained from a maternal host of a fetus. The method comprises contacting the biological sample with an antibody of the invention and detecting the binding of the antibody to a nucleosome in the biological sample. Specific binding of the antibody to the nucleosome is indicative of the presence of fetal nucleic acids and/or fetal cells.

In still another embodiment of the invention, it provides a method for isolating nucleic acids of a fetus. The method comprises isolating nucleic acid from a cervical mucus sample obtained from a maternal host of the fetus by contacting the cervical mucus sample with an antibody of the invention.

In still another embodiment of the invention, it provides a method for enriching nucleic acids of a fetus. The method comprises isolating nucleic acid from a cervical mucus sample obtained from a maternal host of the fetus by contacting the cervical mucus sample with an antibody of the invention.

In still another embodiment of the invention, it provides a method of identifying the genetic composition of a fetus. The method comprises isolating fetal nucleic acid according to a method of the invention and identifying the genetic composition of the fetus based on the isolated fetal nucleic acid.

In still another embodiment of the invention, it provides a composition containing a peptide comprising an amino acid sequence that is at least 80% identical to the sequence of SEQ ID NO: 1 and an adjuvant.

In still another embodiment of the invention, it provides a method of making an antibody that specifically binds to a fetal nucleosome epitope. The method comprises immunizing an animal with a composition containing a peptide comprising an amino acid sequence that is at least 80% identical to the sequence of SEQ ID NO: 1 and an adjuvant.

### DETAILED DESCRIPTION OF THE INVENTION

The present invention is based, in part, on the discovery that nucleosome epitopes can be used as markers for fetal cells or fetal nucleic acids. Accordingly the present invention provides antibodies to these nucleosome epitopes, kits containing these antibodies and methods for using these antibodies to detect or isolate fetal cells and/or fetal nucleic acids.

According to one aspect of the present invention, it provides an antibody that specifically binds to a fetal nucleosome epitope. The term “antibody,” as used herein, includes an antibody binding region, CDR, single chain antibody, chimeric antibody, or humanized antibody. The antibody of the invention can be a monoclonal antibody or polyclonal antibody. The antibody can be any isotype, e.g., IgG, IgM, IgA, IgD, or IgE.

In one embodiment, the fetal nucleosome epitope is any epitope associated with fetal chromatin or nucleosome that is unique to a fetus, or that is distinct from a corresponding maternal epitope. In another embodiment, the fetal nucleosome epitope has a molecular structure (e.g., a primary, secondary, or tertiary structure) that is distinct from that of a corresponding maternal epitope. In yet another embodiment, the fetal nucleosome epitope is more exposed, or more accessible to, the antibody in a fetal nucleosome than in a maternal nucleosome.

In eukaryotes, there are five main classes of histones, which are the chief protein components of chromatin. These are H1 (sometimes called the linker histone), H2A, H2B, H3 and H4. In general two each of the H2A, H2B, H3 and H4 histones assemble to form an octameric nucleosome core particle by wrapping 146 base pairs of DNA around the protein spool. The linker histone H1 and a linker DNA connect the individual nucleosomes like beads on a string. The H3 class of histones consists of four different protein types: the main types, H3.1 and H3.2; the replacement type, H3.3; and the testis specific variant, H3t. Although H3.1 and H3.2 are closely related, only differing at Ser96, H3.1 differs from H3.3 in at least 5 amino acid positions. Further, H3.1 is highly enriched in fetal liver, in comparison to its presence in adult tissues including liver, kidney and heart. In adult human tissue, the H3.3 variant is more abundant than the H3.1 variant, whereas the converse is true for fetal liver. These differences can be exploited by the antibodies of the invention to detect fetal cells and/or fetal nucleic acid in a maternal biological sample that comprises both fetal and maternal cells and/or fetal nucleic acid.

Accordingly, the fetal nucleosome epitope can be an epitope associated with a histone. In one embodiment, the fetal nucleosome epitope is an epitope associated with histone H3. In another embodiment, the fetal nucleosome epitope is an epitope associated with histone H3.1. In yet another embodiment, the fetal nucleosome epitope is an epitope associated with histone H3.1, but not with histone H3.3. In still another embodiment, the fetal nucleosome epitope comprises or consists of an epitope within a peptide with an amino acid sequence: Ser-Ala-Val-Met-Ala-Leu-Gln-Glu-Ala-Cys (SEQ ID NO: 1). In a further embodiment, the fetal nucleosome epitope comprises or consists of an epitope with 2, 3, 4, 5, 6, 7, 8, 9, or 10 amino acid residues of the amino acid sequence of SEQ ID NO: 1. In yet another embodiment, the fetal nucleosome epitope comprises or consists of an epitope within a peptide with an amino acid sequence that is at least 70%, at least 80%, or at least 90% identical to the amino acid sequence of SEQ ID NO: 1. In still another embodiment, the fetal nucleosome epitope is an epitope associated with histone H3.1, and comprises or consists of an epitope within a peptide with an amino acid sequence of SEQ ID NO: 1. Antibodies of the invention include those that compete with antibodies that specifically bind an epitope within a peptide with an amino acid sequence of SEQ ID NO: 1.

The antibodies of the invention can also be targeted to specific conformational or structural features of the histones, especially histone H3.1. For example, the fetal nucleosome epitope can be an epitope associated with a region of histone H3.1 that is more exposed in fetal nucleosomes compared to the corresponding H3.1 in maternal nucleosomes. Differences in post-translational modifications, e.g., methylation or acetylation, that occur in fetal and maternal H3 histones can also be exploited to target fetal H3.1 histones to detect, enrich and/or isolate fetal DNA. Two sets of antibodies, one designed to bind to a specific histone conformation or sequence, and the second designed to bind to a post-translational modification can also be used to isolate fetal nucleic acid.

In one embodiment, the fetal nucleosome epitope does not include an epitope associated with a histone (e.g., histone H3 or H3.1) that comprises a post-translation modification. In another embodiment, the fetal nucleosome epitope does not include an epitope associated with a histone (e.g., histone H3 or H3.1) that comprises a post-translation phosphorylated, methylated, or acetylated amino acid. In yet another embodiment, the fetal nucleosome epitope does not include an epitope associated with a histone (e.g., histone H3 or H3.1) that comprises serine (“S”) residues that have been post-translationally phosphorylated or lysine (“K”) or arginine (“R”) residues that have been methylated or acetylated post translationally. In still another embodiment, the fetal nucleosome epitope does not include an epitope associated with a histone (e.g., histone H3 or H3.1) that comprises a lysine residue at position 4 from the N-terminal end (“K4”) that has been methylated, a lysine residue at position 9 (“K9”) that has been methylated or acetylated, or a serine residue at position 10 (“S10”) that has been phosphorylated.

In general, the antibodies of the invention contain a detectable entity (e.g., a label). Many different detectable labels exist in the art and methods of labeling are well known to the skilled artisan. General classes of labels, which can be used in the present invention, include, but are not limited to, radioactive isotopes, fluorescent, chemiluminescent and bioluminescent molecules, chromophores, enzymes, enzyme substrates, enzyme cofactors, enzyme inhibitors, chromophores, dyes, metal ions, metal sols, ligands (e.g., biotin, avidin, streptavidin, digoxigenin or haptens), etc. Detectable labels include those which fluoresce, including those substances or portions thereof which are capable of exhibiting fluorescence in the detectable range.

Particular examples of labels which may be used in the invention include, but are not limited to, radiolabels, including, but not limited to, ¹²⁵I, ¹³¹I, ³⁵S, ³²P, ¹⁴C and ³H; chemiluminescent compounds including, but not limited to, luminol, isoluminol, theromatic acridinium ester, imidazole, acridinium salt and oxalate ester; bioluminescent compounds including, but not limited to, luciferin, luciferase and aequorin; fluorescent substances including, but not limited to, fluorescein, FITC, rhodamine, phycoerythrin, phycocyanin, allophycocyanin, o-phthaldehyde, Texas red, dansyl chloride, dichlorotriazinylamine fluorescein, green fluorescent protein, and fluorescamin; enzymes including, but not limited to, β-glucuronidase, β-D-glucosidase, β-D-galactosidase, urase, peroxidase, e.g., horseradish peroxidase (HRP), alkaline phosphatase, acetylcholinesterase, glucose oxidase, hexokinase and GDPase, RNase, luciferase, phosphofructokinase, phosphoenolpyruvate carboxylase, aspartate aminotransferase, phosphoenolpyruvate decarboxylase, α-glycerophosphate, asparaginase, glucose-6-phosphate dehydrogenase, glucoamylase, and β-lactamase.

In one embodiment, the antibody of the invention is fluorescently labeled. In another embodiment, the antibody of the invention is fluorescently labeled and the specific binding of the antibody to the fetal cell or fetal nucleic acid is detected by a flow cytometric method, for example, by fluorescent activated cell sorting (FACS).

In yet another embodiment, the antibody of the invention is immobilized on a solid support. The antibody can be immobilized on the solid support either directly, or indirectly, for example, via a linker. Methods for attaching antibodies to solid supports are well known to those skilled in the art and any method now known, or later discovered, can be used. In one embodiment, the solid support is a population of magnetic particles, a particle contained in a column, e.g., a resin column, a surface of a microchannel, a microwell, a plate, an array, a filter, a membrane, or a glass slide.

In another embodiment, the antibody of the invention can be immobilized, or coated on the surface of an apparatus, e.g., a microflow apparatus. An exemplary microflow apparatus comprises an inlet means, an outlet means, and a microchannel arrangement extending between the inlet and outlet means. The microchannel arrangement can be any microchannel capable of providing a randomized flow path for the biological sample. For example, the microchannel arrangement can include a plurality of transverse separator posts that are integral with a base surface of the microchannel and project therefrom. The posts are generally arranged in a pattern capable of providing a randomized flow path. Examples of microflow apparatuses are described in U.S. application Ser. Nos. 11/458,668 and 11/331,988, both of which are incorporated herein in their entirety. The surface of the microchannel arrangement of the microflow apparatus can be coated partially or entirely, with antibodies of the invention.

According to another aspect of the present invention, it provides a kit comprising an antibody of the invention. In one embodiment, the kit further comprises an instruction for using the kit to detect a fetal cell or isolate fetal nucleic acid from a biological sample of a maternal host. In certain embodiments, the kit comprises a microflow apparatus or other solid support coated with an antibody of the invention.

According to yet another aspect of the present invention, it provides a kit suitable for testing the genetic composition of a fetus. The kit comprises an isolated or enriched nucleic acid sample of the fetus that has been isolated or enriched using an antibody of the invention.

According to still another aspect of the present invention, it provides a method for detecting the presence of fetal nucleic acids in a biological sample obtained from a maternal host of the fetus. The method comprises contacting the biological sample with an antibody of the invention and detecting the binding of the antibody to a nucleosome in the biological sample. Specific binding of the antibody to the nucleosome is indicative of the presence of the fetal cell.

The biological sample can be any maternal biological sample, e.g., a bodily fluid or fraction thereof, that is likely to contain a fetal cell or fetal nucleic acid. In one embodiment, the biological sample is a blood, plasma, serum, urine, cervical mucus, amniotic fluid, chorionic villus sample, or a fraction thereof. In another embodiment, the biological sample is blood, plasma, serum, urine, or a fraction thereof. In yet another embodiment, the biological sample is a cervical mucus sample. The biological sample can be obtained from the maternal host of the fetus by any means known or later discovered in the art.

In still another embodiment, the detection step includes detecting specific binding of an antibody of the invention to a cell or a fragment thereof (e.g., an apoptotic body). The specific binding of the antibody to the cell is indicative of the presence of a fetal cell. In general, the detection of specific binding of an antibody of the invention to a nucleosome or to a cell in the biological sample can be carried out using a flow cytometric method. Exemplary flow cytometric methods that can be used, e.g., when the antibody is fluorescently labeled, include, but are not limited to, fluorescent activated cell sorting (FACS).

According to still another aspect of the present invention, it provides a method for isolating or enriching nucleic acids of a fetus. The method comprises isolating nucleic acids from a biological sample obtained from a maternal host of the fetus by contacting the biological sample with an antibody of the invention.

The biological sample can be obtained from the maternal host of the fetus at any time, e.g., during the first, second, or third trimester. In one embodiment, the biological sample is obtained during the first trimester of pregnancy. In another embodiment, the biological sample is obtained during the second trimester. In another embodiment, the biological sample is obtained during weeks 4 through 12, weeks 8 through 12, or weeks 10 through 12 of pregnancy. In yet another embodiment, the biological sample is obtained during weeks 13 through 28, weeks 13 through 24, weeks 13 through 20, weeks 13 through 16, weeks 17 through 20, or weeks 21 through 24 of pregnancy.

The biological sample can be any maternal biological sample as described above. In one embodiment, the biological sample is blood, plasma, serum, urine, cervical mucus, amniotic fluid, chorionic villus sample, or a fraction thereof. In another embodiment, the biological sample is urine, cervical mucus, amniotic fluid, chorionic villus sample, or a fraction thereof. In yet another embodiment, the biological sample is a cervical mucus sample, or a fraction thereof. The cervical mucus sample, e.g., an endocervical mucus sample or a transcervical mucus sample, can be obtained by any technique now known, or later developed, in the art. Such techniques include, but are not limited to, transcervical swabs, endocervical lavage, scrapes, cytobrush, aspiration, intrauterine lavage, or a combination thereof.

The cervical mucus sample can be a fresh sample, e.g., without substantial preservation, or a sample preserved from a fresh sample, e.g., preserved in a suitable aqueous preservation medium, or alternatively, a sample of a medium containing nucleic acids leached from one or more cervical mucus samples. The cervical mucus sample of the present invention can be maintained or stored between about 4° C. and about 20° C., e.g., in a low calcium basal medium.

In one embodiment, the cervical mucus sample is a treated sample, e.g., a fresh or a preserved sample treated with a suitable reagent(s) to facilitate mucus dissolution which in turn, assists in isolation of nucleic acid components from the sample. For example, the cervical mucus sample of the present invention can be a sample treated with mucolytic agent(s) or mucinase(s), e.g., N-acetyl-L-cysteine, L-cysteine, dithiothreitol (DTT), brornhexine hydrochloride, and any of the hyaluronidases, including hyaluronate lyase, hyaluronoglucosarninidase, and hyaluronglucuronidase. In another example, the cervical mucus sample of the present invention is a sample treated with enzyme(s), e.g., sugar hydrolysis enzyme(s), such as β-galactosidase or invertase; proteinase; pepsin; or combinations thereof.

In another embodiment, the biological sample obtained from the maternal host of the fetus is subjected to apoptosis-inducing treatment, or, is treated to facilitate the release of fetal nucleic acid from intact fetal cells, prior to being contacted by the antibody. For example, the biological sample can be a sample treated with reagents, or subjected to high pH, shearing, or heat shock. Reagents such as an etoposide, including, but not limited to, VP16, and other comparable enzymes can be used to facilitate the release of fetal nucleic acid.

In yet another embodiment, the biological sample is treated to enrich fetal nucleic acid and/or reduce maternal nucleic acid content. For example, the biological sample can be treated to reduce or degrade any nucleic acid, e.g., DNA that is characteristic of maternal DNA. One example of such a nucleic acid is hypermethylated maternal DNA. Any means to reduce, degrade, or selectively remove hypermethylated maternal DNA can be used including, without any limitation, methylation specific restriction enzymes such as McrBC (BioLabs), antibodies specific for hypermethylated maternal DNA such as anti-5′-methyl-cytosine antibodies and/or anti-methylCpG binding protein-2 (MeCP2) antibodies, or ligands or proteins such as MeCP2 that specifically bind methylated CpG islands in maternal DNA.

Alternatively fetal nucleic acid can be enriched using markers specific for fetal nucleic acids. For example, hypomethylated maspin DNA can be used as a marker for fetal DNA. In one embodiment, total DNA from the biological sample can be treated with sodium bisulfite which could induce chemical changes in the hypomethylated fetal DNA, whereby unmethylated cytosine of fetal DNA is converted into uridine (U). Such change can be used to preferentially isolate or enrich fetal DNA, e.g., to preferentially amplify fetal DNA containing uridine(s) converted from cytosine(s).

According to still another aspect of the present invention, it provides a method of identifying the genetic composition of a fetus. The method comprises isolating fetal nucleic acid according to a method of the invention and identifying the genetic composition of the fetus based on the isolated fetal nucleic acid.

The genetic composition of the fetus can be indicative of the gender of the fetus, or of a condition or disorder in the fetus. In one embodiment, the genetic composition includes, without any limitation, monosomy, partial monosomy, trisomy, partial trisomy, chromosomal translocation, chromosomal duplication, chromosomal deletion or microdeletion, and chromosomal inversion.

In general, the term “monosomy” refers to the presence of only one chromosome from a pair of chromosomes. Monosomy is a type of aneuploidy. Partial monosomy occurs when the long or short arm of a chromosome is missing. Common human genetic disorders arising from monosomy include: X0, only one X chromosome instead of the usual two (XX) seen in a normal female (also known as Turner syndrome); cri du chat syndrome, a partial monosomy caused by a deletion of the end of the short p (from the word petit, French for small) arm of chromosome 5; and 1p36 Deletion Syndrome, a partial monosomy caused by a deletion at the end of the short p arm of chromosome 1.

In contrast, the term “trisomy” refers to the presence of three, instead of the normal two, chromosomes of a particular numbered type in an organism. Thus the presence of an extra chromosome 21 is called trisomy 21. Most trisomies, like most other abnormalities in chromosome number, result in distinctive birth defects. Many trisomies result in miscarriage or death at an early age. A partial trisomy occurs when part of an extra chromosome is attached to one of the other chromosomes, or if one of the chromosomes has two copies of part of its chromosome. A mosaic trisomy is a condition where extra chromosomal material exists in only some of the organism's cells. While a trisomy can occur with any chromosome, few babies survive to birth with most trisomies. The most common types that survive without spontaneous abortion in humans include: Trisomy 21 (Down syndrome); Trisomy 18 (Edwards syndrome); Trisomy 13 (Patau syndrome); Trisomy 9; Trisomy 8 (Warkany syndrome 2); and Trisomy 16 (which is the most common trisomy in humans, occurring in more than 1% of pregnancies, although it usually results in spontaneous miscarriage in the first trimester). Trisomy involving sex chromosomes include: XXX (Triple X syndrome); XXY (Klinefelter's syndrome); and XYY (XYY syndrome).

In another embodiment, the genetic composition includes allele or gene abnormalities, e.g., one or more mutations such as point mutations, insertions, deletions in one or more genes.

In yet another embodiment, the genetic composition includes, without any limitation, one or more polymorphism patterns or genetic markers, e.g., short tandem repeat sequences (STRs), single nucleotide polymorphisms (SNPs), etc.

In still another embodiment, the genetic composition is indicative of a disease or disorder. Examples of diseases or disorders include, but are not limited to, Cystic Fibrosis, Sickle-Cell Anemia, Phenylketonuria, Tay-Scahs Disease, Adrenal Hyperplasia, Fanconi Anemia, Spinal Muscularatrophy, Duchenne's Muscular Dystrophy, Huntington's Disease, Beta Thalassaemia, Myotonic Dystrophy, Fragile-X Syndrome, Down Syndrome, Edwards Syndrome, Patau Syndrome, Klinefelter's Syndrome, Triple X syndrome, XYY syndrome, Trisomy 8, Trisomy 16, Turner Syndrome, Robertsonian translocation, Angelman syndrome, DiGeorge Syndrome, Wolf-Hirschhorn Syndrome, RhD Syndrome, Tuberous Sclerosis, Ataxia Telangieltasia, and Prader-Willi syndrome.

In a further embodiment, the genetic composition includes any genetic condition or abnormality that is not uniquely associated with a Y chromosome.

In yet another embodiment, the genetic composition includes any genetic condition corresponding to or associated with paternity of the fetus.

The genetic tests of the present invention may use the isolated nucleic acid sample of the invention either directly or as templates for “amplification-based” genetic composition testing assays. These assays are well known in the art and include, without any limitation, polymerase chain reaction (“PCR”), real-time polymerase chain reaction (“RT-PCR”), ligase chain reaction (“LCR”), self-sustained sequence replication (“35R”) also known as nucleic acid sequence based amplification (“NASBA”), Q-B-Replicase amplification, rolling circle amplification (“RCA”), transcription mediated amplification (“TMA”), linker-aided DNA amplification (“LADA”), multiple displacement amplification (“MDA”), and invader and strand displacement amplification (“SDA”).

According to still another aspect of the present invention, it provides a composition containing a peptide comprising an amino acid sequence that is at least 70%, at least 80%, or at least 90% identical to the sequence of SEQ ID NO: 1, and an adjuvant. In one embodiment, the peptide comprises an amino acid sequence that is 100% identical to the sequence of SEQ ID NO: 1. Suitable adjuvants include, but are not limited to, Freund's complete adjuvant, Freund's incomplete adjuvant, substitutes for Freund's complete adjuvant, Alum, ethylene vinyl acetate copolymer, L-tyrosine, manide-oleate, saponin/cholesterol micelles, or nitrocellulose (i.e., nitrocellulose to which peptide antigen is absorbed).

According to yet another aspect of the present invention, it provides a method of making an antibody that specifically binds to a fetal nucleosome epitope. The method comprises immunizing an animal with a composition containing a peptide comprising an amino acid sequence that is at least 80% identical to the sequence of SEQ ID NO: 1 and an adjuvant. The animal can be, for example, a mammal (e.g., a mouse, rat, guinea pig, rabbit, goat, horse, etc.). In certain embodiments, the method comprises obtaining cells (e.g., spleen cells) from an immunized animal, generating monoclonal antibodies, and screening such monoclonal antibodies for fetal nucleosome-specific binding. In other embodiments, the method comprises obtaining polyclonal anti-serum from an immunized animal and, optionally, purifying fetal nucleosome-specific antibodies from the polyclonal anti-serum.

### EXAMPLES

The following examples are intended to illustrate, but not to limit, the invention in any manner, shape, or form, either explicitly or implicitly. While they are typical of those that might be used, other procedures, methodologies, or techniques known to those skilled in the art may alternatively be used.

### Example 1

A cervical mucus sample is obtained by swabbing or lavage from a pregnant woman early in the second trimester (or late in the first trimester) of pregnancy. The cervical mucus sample on the collection device is placed in a tube containing an aqueous buffer, stored at about 4° C., and transported to a processing laboratory while maintaining the sample temperature at about 4° C. The maintenance of a 4° C. environment may be achieved using a suitable ice pack or the like.

Upon arrival at the processing laboratory, the sample is checked in and forwarded to a laboratory technician for processing. The sample is allowed to warm to room temperature for subsequent processing. Next, the sample may be placed on a rocker in a 37° C. incubator for approximately 15 to 30 minutes to release DNA trapped in mucus.

Mucus may be further dissolved with a mucinase for the purpose of releasing DNA, and the sample may also be subjected to conditions, such as chemical treatment and the like, as well known in this art, to induce apoptosis to release fetal nucleosomes. The collection device is removed and the aqueous buffer is filtered with, for example, a 0.22 gm filter, to remove contaminants including maternal cells and sperm cells. Alternatively, the liquid from the sample may be centrifuged to pellet in order to separate free DNA from the high molecular weight mucus and cells. DNAse I is optionally added to the sample in aqueous buffer to remove unprotected high molecular weight DNA.

The cleaned, concentrated sample is then run through a microflow device of the type disclosed in U.S. patent application Ser. No. 11/038,920, having a collection region with a multitude of randomly positioned posts which is prepared by attaching histone-specific antibodies within its post-containing collection region. The antibodies may be designed to couple with the decapeptide having the amino acid residue sequence: Ser-Ala-Val-Met-Ala-Leu-Gln-Glu-Ala-Cys.

The treated mucus sample, prepared as above, travels slowly through the microflow device, drawn by a vacuum pump. The antibodies couple to and sequester chromatin in the sample containing fetal histone 3.1. by coupling to the exposed unique sequence. The microflow device is then washed with buffers, and washing is repeated 2-3 times to remove any nonspecifically bound biologic material that may have been present in the cervical mucus.

Antibodies which are designed to couple with a post-translational modification, such as glycosylation, phosphorylation, and/or methylation on fetal nucleic acid can also be used. Alternatively, two sets of antibodies are used that are designed to couple with a specific histone conformation and with a post-translational modification as described above.

The biological material sequestered by the antibodies is released from the microflow device and subjected to purification to remove salts that might be present. The DNA is then extracted from the sample using a commercial DNA extraction kit, such as those available from Roche.

The DNA is then concentrated to an appropriate volume, i.e. 20-50 μl, and analysis is carried out to detect chromosome Y-specific sequences using Realtime PCR or fluorescence-based PCR. The results of the analysis show positive detection of male Y-sequences in the DNA, which is evidence of fetal DNA being present in the biological material that is sequestered by the antibodies. The absence, to any significant extent, of maternal DNA in the sequestered DNA material is shown using quantitative Realtime PCR of two loci. For example, showing that Y-levels are equal to at least one-half the level of a second ubiquitous sequence (e.g., (β-globin) is evidence that most or nearly all of DNA is of fetal origin. Alternatively, a determination of the relative proportion of H3.1 to H3.3 in the DNA may be used to show that the relative proportions which exist are such as would be present in nucleosomes of fetal origin, which would confirm the fetal origin of the DNA.

Following such confirmation, analysis of the DNA for aneuploidies or other genetic disorders or conditions that might suggest pregnancy complications may be carried out with confidence. The isolated fetal DNA may be subjected to molecular DNA sequencing or polymorphic DNA sequence analysis. Such analysis may utilize Realtime PCR to quantify DNA levels that are associated with specific DNA sequences to screen for aneuploidies or may use PCR to amplify the DNA and then incubate it with mutation microarrays or gene-specific microarrays.

All patents and publications referred to herein are expressly incorporated by reference in their entirety.

Although the invention has been described with reference to the presently preferred embodiments, it should be understood that various modifications can be made without departing from the spirit of the invention. Accordingly, the invention is limited only by the following claims.

What is claimed:

1. An antibody that specifically binds to a fetal nucleosome epitope.

2. The antibody of claim 1, wherein the fetal nucleosome epitope is an epitope associated with histone H3.

3. The antibody of claim 1, wherein the fetal nucleosome epitope is an epitope associated with histone H3.1.

4. The antibody of claim 1, wherein the fetal nucleosome epitope is an epitope associated with a region of histone H3.1 that is more exposed in fetal nucleosomes than in maternal nucleosomes.

5. The antibody of claim 1, wherein the fetal nucleosome epitope is an epitope associated with histone H3.1, but not with histone H3.3.

6. The antibody of claim 1, wherein the fetal nucleosome epitope is an epitope within a peptide with an amino acid sequence of SEQ ID NO: 1.

7. An antibody that specifically binds to a fetal nucleosome epitope, wherein the antibody competes with the antibody of claim 6 in its binding to a peptide containing the epitope.

8. The antibody of claim 1, wherein the fetal nucleosome epitope is an epitope within a peptide with an amino acid sequence that is at least 90% identical to the amino acid sequence of SEQ ID NO. 1.

9. The antibody of claim 1, wherein the antibody contains a detectable entity.

10. The antibody of claim 1, wherein the antibody is fluorescently labeled.

11. The antibody of claim 1, wherein the antibody is immobilized on a solid support.

12. A kit comprising the antibody of claim 1.

13. The kit of claim 12, further comprising an instruction for using the kit to detect a fetal cell or isolate fetal nucleic acid from a biological sample of a maternal host.

14. A kit suitable for testing genetic composition of a fetus comprising an isolated nucleic acid sample of a fetus, wherein the sample is isolated by using the antibody of claim 1.

15. A method for detecting the presence of fetal nucleic acids in a biological sample comprising:
contacting the biological sample with the antibody of claim 1, and
detecting the binding of the antibody to a nucleosome in the biological sample,
wherein specific binding of the antibody to a nucleosome is indicative of the presence of fetal nucleic acids.

16. The method of claim 15, wherein the biological sample is a blood, plasma, serum, urine, cervical mucus, amniotic fluid, or chorionic villus sample.

17. The method of claim 15, wherein the biological sample is a cervical mucus sample.

18. The method of claim 15, wherein detection includes detecting specific binding of the antibody to a cell and wherein specific binding of the antibody to the cell is indicative of the presence of a fetal cell.

19. The method of claim 15, wherein detection is carried out using a flow cytometric method.

20. The method of claim 15, wherein detection is carried out using fluorescent activated cell sorting (FACS) and wherein the antibody is fluorescently labeled.

21. A method for isolating nucleic acid of a fetus comprising:
isolating nucleic acid from a biological sample obtained from a maternal host of the fetus using the antibody of claim 1.

22. The method of claim 21, wherein the biological sample is urine, cervical mucus, amniotic fluid, or chorionic villus sample.

23. The method of claim 21, wherein the antibody is immobilized on a solid surface.

24. The method of claim 21, wherein the biological sample is subjected to apoptosis-inducing treatment prior to being contacted by the antibody.

25. The method of claim 21, wherein the biological sample is subjected to apoptosis-inducing treatment prior to being contacted by the antibody, wherein the apoptosis-inducing treatment comprises: chemical treatment, high pH, shearing, or heat shock.

26. The method of claim 21, wherein the biological sample is obtained during the first trimester.

27. The method of claim 21, wherein the biological sample is a cervical mucus sample, and wherein the cervical mucus sample is obtained by transcervical swabs, endocervical lavage, cytobrush, aspiration, intrauterine lavage, or a combination thereof.

28. The method of claim 21, wherein the biological sample is a cervical mucus sample, and wherein the cervical mucus sample is treated with a mucolytic agent prior to being contacted by the antibody.

29. A method of identifying the genetic composition of a fetus comprising:
isolating fetal nucleic acid according to the method of claim 21; and
identifying the genetic composition of the fetus based on the isolated fetal nucleic acid.

30. The method of claim 29, wherein the genetic composition is selected from the group consisting of monosomy, partial monosomy, trisomy, partial trisomy, chromosomal translocation, chromosomal duplication, chromosomal deletion or microdeletion, and chromosomal inversion.

31. The method of claim 29, wherein the genetic composition is indicative of a disease or disorder selected from the group consisting of Cystic Fibrosis, Sickle-Cell Anemia, Phenylketonuria, Tay-Scahs Disease, Adrenal Hyperplasia, Fanconi Anemia, Spinal Muscularatrophy, Duchenne's Muscular Dystrophy, Huntington's Disease, Beta Thalassaemia, Myotonic Dystrophy, Fragile-X Syndrome, Down Syndrome, Edwards Syndrome, Patau Syndrome, Klinefelter's Syndrome, Triple X syndrome, XYY syndrome, Trisomy 8, Trisomy 16, Turner Syndrome, Robertsonian translocation, Angelman syndrome, DiGeorge Syndrome, Wolf-Hirschhorn Syndrome, RhD Syndrome, Tuberous Sclerosis, Ataxia Telangieltasia, and Prader-Willi syndrome.

32. A composition containing a peptide comprising an amino acid sequence that is at least 80% identical to the sequence of SEQ ID NO: 1 and an adjuvant.

33. The composition of claim 32, wherein the amino acid sequence is 100% identical to the sequence of SEQ ID NO: 1.
